(19) United States
(12) Patent Application Publication
(10) Pub. No.: US 2022/0008179 A1
(43) Pub. Date: Jan. 13, 2022

(54) CHEEK RETRACTOR DEVICE

(71) Applicant: Élie Stephan, Outremont (CA)

(72) Inventor: Élie Stephan, Outremont (CA)

(21) Appl. No.: 17/369,601

(22) Filed: Jul. 7, 2021

Related U.S. Application Data
(60) Provisional application No. 63/049,327, filed on Jul. 8, 2020.

(51) Int. Cl. A61C 17/10

(52) U.S. Cl. CPC A61C 17/10

(57) ABSTRACT

A dental apparatus for insertion within a person's mouth, in combination with a suction device is provided. The dental apparatus includes a cheek retractor having a pair of wings shaped and adapted to conform to a periphery of a person's mouth and a linking element extending between the pair of arcuate wings. The linking element enabling movement of the pair of wings relative to one another and to bias the arcuate wings into a spaced-apart position, where the arcuate wings are adapted to apply an outwardly oriented force on the cheeks to maintain the cheeks and lips retracted. The apparatus also includes a connection assembly having one or more tubing elements connected to the cheek retractor proximate the arcuate wings and being operatively connected to the suction device, with the suction device being operable to provide suction to a surrounding area of the cheek retractor via the tubing elements.

### CROSS-REFERENCE TO RELATED APPLICATIONS

This application claims priority under 35 USC § 119(e) of U.S. Provisional Application No. 63/049,327, filed Jul. 8, 2020, entitled “IMPROVED CHEEK RETRACTOR DEVICE”, the entirety of which is hereby incorporated by reference.

### TECHNICAL FIELD

The technical field generally relates to dental tools for general and/or specialized dentistry, and more particularly to a cheek retracting device adapted to provide improved suction of aerosol particles during dental procedures.

### BACKGROUND

Various dental procedures can directly or indirectly create airborne particles, such as dust, droplets and/or aerosols in and around the working environment of the operator, such as a dentist, a dental hygienist, or a surgeon. These airborne particles can remain suspended in the air for extended periods of time and can carry bacteria and viruses, among others, as it can contain the patient's saliva or organic tissue, for example. Therefore, a patient being operated on can potentially contaminate the dentist, dental hygienist or surgeon, the supporting team, or any subsequent patient using the same operating room/chair.

Multiple devices provide suction to reduce the dispersion of the airborne particles. However, these devices can have an excessive size, restrict access for the operator/user or require a user (e.g., the dentist, his/her assistant or the patient) to manually hold and orient the device.

Thus, it would be particularly useful to be able to provide a new device, which, by virtue of its design and components, would be able to overcome or at least minimize some of the known drawbacks associated with currently available, for example.

### SUMMARY

According to an aspect, a cheek retracting device for maintaining cheeks and lips retracted is provided. The cheek retracting device includes a cheek retractor having a pair of wings adapted to engage a mouth of a person on either side thereof, where each wing has an inner portion adapted to extend along an inner side of the cheek, and an outer portion adapted to extend over a periphery of the mouth and engage an outer side of the cheek. The cheek retractor also includes a resilient linking element connected to and extending between the pair of wings, the resilient linking element being adapted to enable movement of the pair of wings relative to one another and bias the wings into a spaced-apart position to maintain the cheeks and lips retracted when the wings are engaged therewith. The cheek retracting device further includes a connection assembly adapted to connect the cheek retractor to a suction device, the connection assembly having one or more hollow tubes operatively connected to and extending between the suction device and the cheek retractor to enable suction of airborne particles proximate at least one of the pair of wings and the resilient linking element.

According to a possible embodiment, the resilient linking element is connected to and extends between the outer portions of the pair of wings.

According to a possible embodiment, the one or more hollow tubes comprises a main tube operatively connected to the suction device and one or more secondary tubes having a first end fluidly connected to the main tube, the one or more secondary tubes having a second end provided with a suction aperture positioned proximate one of the wings, where, during operation of the suction device, the suction aperture provides suction to a surrounding area.

According to a possible embodiment, the connection assembly comprises a generally conical and rigid connector adapted to fluidly connect the main tube to the suction device.

According to a possible embodiment, an outer diameter of the rigid connector is substantially the same as the inner diameter of the main tube.

According to a possible embodiment, the connection assembly comprises a junction point having a plurality of connection ports, the main tube and secondary tubes being connected to respective connection ports to establish fluid communication between the main tube and the secondary tubes.

According to a possible embodiment, the connection assembly comprises one or more tube connectors adapted to connect the one or more secondary tubes to the cheek retractor.

According to a possible embodiment, each tube connector comprises a back surface connectable to the cheek retractor, and further comprises one or more clasping elements extending from the back surface and configured to hold the second end of the secondary tubes.

According to a possible embodiment, the back surface is connected to the resilient linking element proximate one of the wings, and wherein the clasping element is adapted to hold the second end of the secondary tube proximate the mouth of the person.

According to a possible embodiment, the clasping elements are generally circular, and wherein an inner diameter of the clasping elements is substantially the same as an outer diameter of the secondary tube.

According to a possible embodiment, the tube connector comprises an elongate body connected to and extending along the outer portion of the wing, the elongate body being hollow and comprising a plurality of apertures dispersed therealong, the secondary tube being fluidly connected to the tube connector to provide suction to the surrounding environment via each one of the plurality of apertures.

According to a possible embodiment, the tube connectors comprise a substantially conical connection port, and wherein the secondary tube is fluidly connectable to the connection port.

According to a possible embodiment, at least one of the wings of the cheek retractor includes a hole defined through the outer portion and being adapted to receive one of the secondary tubes to position the second end thereof proximate the mouth of the person.

According to a possible embodiment, the secondary tubes are positioned proximate the wings of the cheek retractor in a manner such that the second end of the secondary tube and the edge of the wing define an angle between about 75 and 100 degrees.

According to a possible embodiment, the hollow tubes of the connection assembly are made of silicone.

According to a possible embodiment, the hollow tubes are releasably connectable to the cheek retractor.

According to another aspect, there is provided a dental apparatus for insertion within a person's mouth, in combination with a suction device. The dental apparatus includes a cheek retractor having a pair of arcuate wings shaped and adapted to conform to at least a portion of a periphery of a person's mouth; and a resilient linking element connected to and extending between the pair of arcuate wings, the resilient linking element being adapted to enable movement of the pair of wings relative to one another, where the arcuate wings are displaceable toward one another for insertion within the mouth, the resilient linking element being adapted to bias the arcuate wings to a spaced-apart position, where the arcuate wings are adapted to apply an outwardly oriented force on the cheeks to maintain the cheeks and lips retracted; and a connection assembly having one or more tubing elements connected to the cheek retractor proximate the arcuate wings, the tubing elements being operatively connected to the suction device, with the suction device being operable to provide suction to a surrounding area of the cheek retractor via the tubing elements.

According to a possible embodiment, the tubing elements have a free end positioned proximate the arcuate wings, and wherein the free end comprises a suction port configured to provide suction to the surrounding area of the suction port during operation of the suction device.

According to a possible embodiment, the free end is adapted to extend through a thickness of the arcuate wing.

According to a possible embodiment, the tubing elements are releasably connected to the cheek retractor.

### BRIEF DESCRIPTION OF THE DRAWINGS

FIG. 1 is a front view of a cheek retractor provided with a connection assembly, according to a possible embodiment.

FIG. 2 is a front view of the cheek retractor shown in FIG. 1, showing a linking element extending between a pair of wings, according to an embodiment.

FIG. 3 is a sectional view of a tube connection component, according to an embodiment.

FIG. 4 is a front view of the cheek retractor shown in FIG. 1, showing a pair of secondary tubes connected to the cheek retractor proximate the pair of wings.

FIG. 5 is a right side view of the cheek retractor shown in FIG. 4.

FIG. 6 is a left side view of a cheek retractor having secondary tubes connected thereto via a tube connector, according to an embodiment.

FIG. 7 is a cross-sectional view of the tube connector shown in FIG. 6, showing a secondary tube secured via clasping elements, according to an embodiment.

FIG. 8 is a front view of the embodiment of the cheek retractor shown in FIG. 6.

FIG. 9 is a perspective view of the cheek retractor shown in FIG. 2, with secondary tubes connected thereto with respective components, according to a possible embodiment.

FIG. 10 is a top view of a cheek retractor according to an embodiment, showing holes defined in the wings of the cheek retractor.

FIG. 11 is back view of the cheek retractor shown in FIG. 10, showing a pair of secondary tubes connected to the holes, according to a possible embodiment.

FIGS. 12 and 13 are front and side views of an alternative embodiment of the cheek retractor, showing additional apertures defined proximate the wing of the cheek retractor.

FIG. 14 is a front view of an alternate embodiment of the cheek retractor, showing a hollow linking element connected to a single suction tube.

### DETAILED DESCRIPTION

As will be explained below in relation to various embodiments, a cheek retracting device adapted to provide suction of airborne particles is provided. The cheek retracting device includes a cheek retractor and a connection assembly adapted to connect one or more component to the cheek retractor. In the context of the present disclosure, the connected components relate to dentistry tools and equipment, although it will be appreciated that other types of tools can be used. In some embodiments, the dentistry equipment can include various tubing operatively connected to a suction device, or suctioning mechanism, in order to control airborne particles in and around a patient's mouth during dental procedures, for example.

The cheek retracting device can be inserted in a patient's mouth and is operable the hold the patient's cheeks in a retracted position, thereby providing ease of access to the interior of the mouth (e.g., to clean the teeth, operate in an area inside the mouth, etc.). The cheek retracting device includes cheek-engaging components held in a spaced-apart configuration via a resilient linking member. The cheek-engaging components can be displaced relative to one another, for example via manual application of force on the cheek-engaging components toward each other. This displacement facilitates insertion of the cheek retracting device into the patient's mouth. The resilient linking member is configured to bias the cheek-engaging components back into their initial position (e.g., in the spaced-apart configuration), thereby applying an outwardly oriented force on the patient's cheeks to maintain them in the retracted position.

The cheek retracting device is used in combination with a suction mechanism or device, and is adapted to maintain the cheeks of the patient in the retracted position while providing suction to the area within and/or around the patient's mouth. As will be described further below, the suction device can be adapted to provide suction at one or more locations of the cheek retracting device, such as proximate the cheek-engaging components, along the resilient linking member or a combination thereof, for example. It is thus appreciated that, once the cheek retracting device is installed for operation, the patient's mouth can be maintained open, with the cheeks in the retracted position, and suction can be provided in and around the patient's mouth (e.g., in and around the working environment of a dentist) with minimal intervention from a person, or without any intervention what so ever.

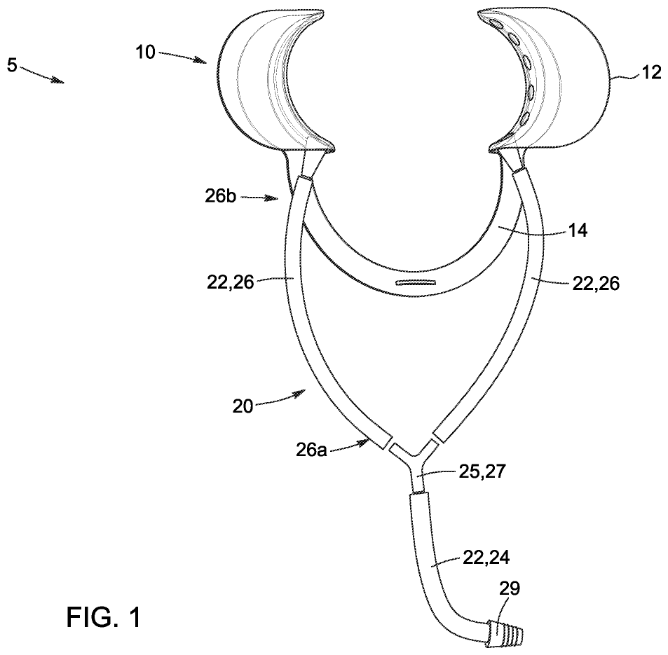

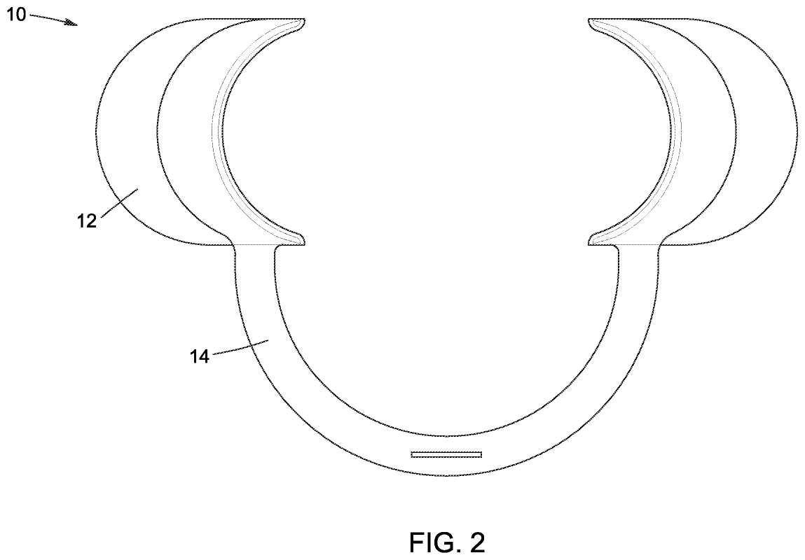

Referring to FIGS. 1 and 2, a possible embodiment of a cheek retracting device 5 is shown. The cheek retracting device 5 includes a cheek retractor 10 configured to retract a patient's cheeks to create a cleared operating area for the user. In the present embodiment, the user can be a surgeon, a hygienist or a dentist, and the operating area can include the area in and around the patient's mouth. The cheek retractor 10 includes two wings 12 connected to one another via a linking member 14 extending therebetween. Each wing 12 is shaped and configured to respectively engage a cheek of the patient to position the cheeks and lips in a retracted position to keep the mouth open and facilitate access to the teeth to perform dental work, for example.

As seen in FIG. 1, the wings 12 are generally arcuate, and can be C-shaped to conform to the cheeks of the patient along a periphery of the mouth (e.g., along the sides of the lips). The wings 12 are sized and adapted to maintain the cheeks retracted, for example, to facilitate access to the back of the mouth, and also maintain the lips retracted to facilitate access to the teeth, such as the front teeth. The wings 12 can extend between the lower lip and the upper lip on either side of the mouth while keeping a central portion of the mouth (e.g., of the lips) free from contact. In alternate embodiments, it is noted that the wings can be sized and adapted to extend from either side of the mouth and come in contact with one another around the mouth. In yet another possible embodiment, the cheek retractor can be provided with a single wing 12 extending between the left and right-sides of the mouth along either one of the upper and lower lips, or generally around the entire periphery of the mouth (e.g., by 360 degrees).

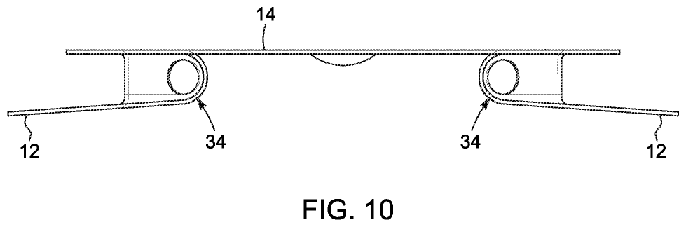

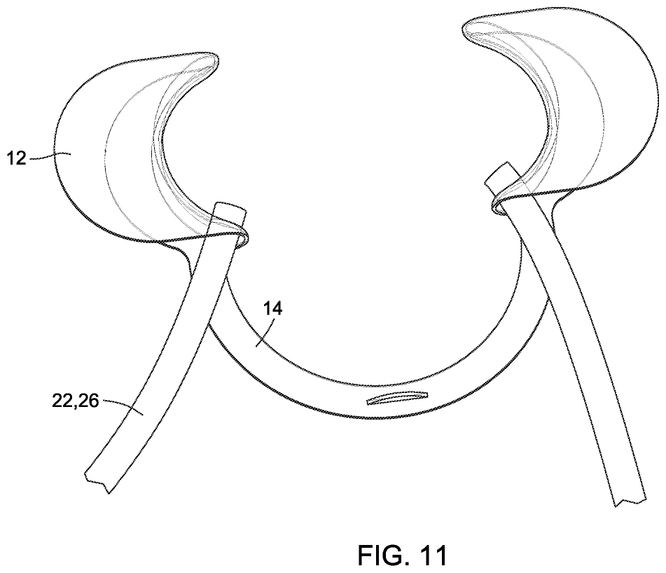

The wings can include an inner portion 13 configured to engage the inside of the patient's mouth, such as the inner side of the cheek, and an outer portion 15 configured to extend over the periphery of the mouth and engage the outer side of the cheek. As seen, in FIG. 10, the inner and outer portions 13, 15 can be connected to one another via a central portion 17 extending therebetween. The central portion 17 can be arcuate which can improve comfort of the patient during use of the cheek retractor 10 and facilitate extension of the outer portion 15 over the periphery of the mouth, although other configurations are possible and may be used. In this embodiment, the inner portion 13 is illustratively larger than the outer portion 15, which can further improve comfort of the patient. However, it is appreciated that other configurations are possible, such as having the inner and outer portions be substantially the same size, for example.

The linking member 14 can also be arcuate, as seen in FIG. 1, to link the wings 12 while also avoiding creating obstructions across the open mouth of the patient. The linking member 14 can be a resilient element configured to allow the wings to be displaced relative to one another (e.g., moved closer and/or away relative to one another), and return to their initial position once the wings are released. It will be understood that the resilient linking element 14 allows the cheek retractor 10 to retract the cheeks of the user by creating an outwardly orientated force on the cheeks as the linking element 14 maintains the wings 12 separated on either sides of the patient's mouth. It should be noted that, although the linking element is shown as a generally singular piece extending between the wings 12, the linking element 14 can include two or more portions connected to each other and extending between the wings 12. In this embodiment, the linking element 14 is connected to a bottom section of the wings and is adapted to extend downwardly proximate or below the patient's chin (i.e., away from the open mouth of the patient). It is appreciated that other configurations of the linking element are possible, such as a linking element extending from the top sections of the wings, and extend proximate, over or above the patient's nose, for example.

In some embodiments, the cheek retractor 10 is operatively connected to a suction device, such as a vacuum, to capture (i.e., suck up) saliva and other particles which can travel through the air, thus greatly reducing the number of particles travelling/suspended in the surrounding air. The cheek retractor 10 can be used to maintain the operation area (e.g., the mouth of the patient) clear by keeping the patient's mouth open, and simultaneously create suction, via the suction device, to create a cleaner and safer work environment during various procedures. It is appreciated that the wings 12 are configured to remain in place without being held (e.g., via the resilient linking element 14 and their geometrical configuration) such that the user (e.g., dentist, surgeon or other) can work without having to intervene or handle additional equipment to keep the mouth open, or to maintain a clean and substantially dry work environment.

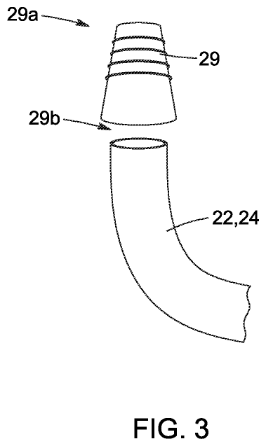

Now referring to FIGS. 3 to 5, in addition to FIGS. 1 and 2, in some embodiments, the cheek retracting device 5 includes a connection assembly 20 configured to connect the cheek retractor 10 to the suction device 50 (seen in FIG. 1). The connection assembly 20 can include one or more hollow tubes 22 operatively connecting the suction device 50 to the cheek retractor 10 to create suction proximate the wings 12, and thus proximate the mouth opening of the patient. As seen in FIG. 1, the connection assembly 20 can include a main tube 24 connectable to the suction device 50, and a pair of secondary tubes 26. The secondary tubes 26 can have a first end 26a, such as a proximal end, in fluid communication with the main tube 24, and a second end 26b, such as a distal end, connected to the cheek retractor 10 to provide suction proximate the mouth opening (e.g., when installed on a patient). However, it is appreciated that any other number of tubes and/or tube configurations are possible and may be used in the context of the present invention. For example, a third secondary tube can be positioned to provide additional suction proximate the patient's mouth, or a single secondary tube provided with multiple branches connected to respective components or locations of the cheek retractor 10.

The main tube 24 and secondary tubes 26 can be fluidly connected together at a junction point 25. In this embodiment, the junction point 25 is a separate component having three connection ports, each connection port being adapted to have one of the tubes (e.g., one of the main tube 24 and secondary tubes 26) connect thereto. As seen in FIG. 1, the junction point 25 can be a Y-shaped connector 27 to which each tube can be connected to such that fluids (e.g., saliva and/or other airborne substances/particles) can be suctioned from the patient's mouth via the secondary tubes 26. It should be understood that the fluids are routed from the secondary tubes 26 to the main tube 24 via the Y-shaped connector 27, and then routed to the suction device 50. In some embodiments, the Y-shaped connector 27 can be replaced by a control valve (not shown) configured to control the flowrate of fluids through the tubes 22. For example, the control valve can be operable to have a single one of the secondary tubes 26 provide suction proximate the patient's mouth, both secondary tubes 26 provide suction, or none of the secondary tubes 26 provide suction.

In the exemplary embodiment of FIG. 1, the main tube 24 and secondary tubes 26 are all separate components connected together via the junction point 25. However, it is appreciated that a single tubing element having multiple branches can be used to suction fluids away from the patient's mouth. In such embodiments, it is understood that the junction point 25 is integrally formed with the tubing of the connection assembly 20. Alternatively, or additionally, more than one secondary tube 26 can be connected to a single one of the wings 12 such that suction is provided at various locations along said wing 12, and thus at various locations along the opening of the patient's mouth. It should be noted that the junction point 25 can include more than three connection ports to accommodate a connection assembly 20 having more than two secondary tubes 26 and/or more than one main tube 24, for example.

In addition, in some embodiments, the main tube 24 can be provided with a rigid connector 29 for engaging the suction device. As seen in FIG. 3, the rigid connector 29 can have any suitable shape and size, such as a conical shape, whereas the main tube 24 connects to a first side of the rigid connector 29a having a first diameter, and the second side of the rigid connector 29b having a second diameter. In this embodiment, the second diameter is smaller than the first diameter and is adapted to be connected to the suction device 50, such as to a port thereof. For example, the first diameter can be between about 11 mm and 12 mm, and the second diameter can be between 9 mm and 10 mm. However, it is appreciated that other configurations are possible. It is noted that the openings of the rigid connector 29 can have any suitable shape. For example, the opening of the first side 29a can be substantially oval, while the opening of the second side 29b can be substantially circular, or vice-versa.

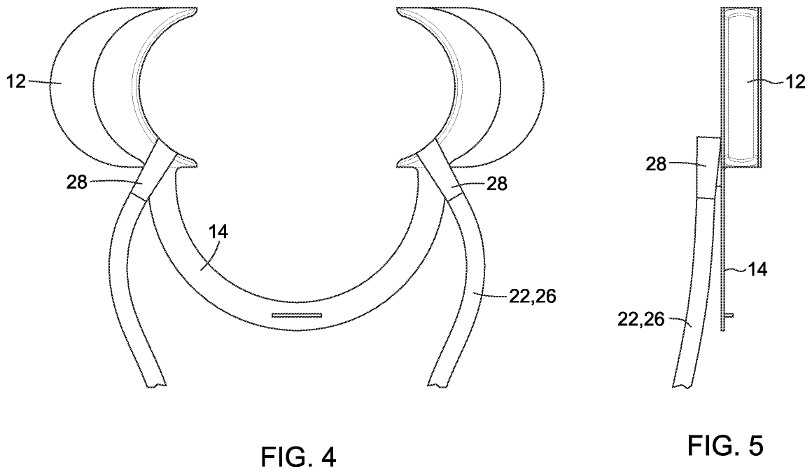

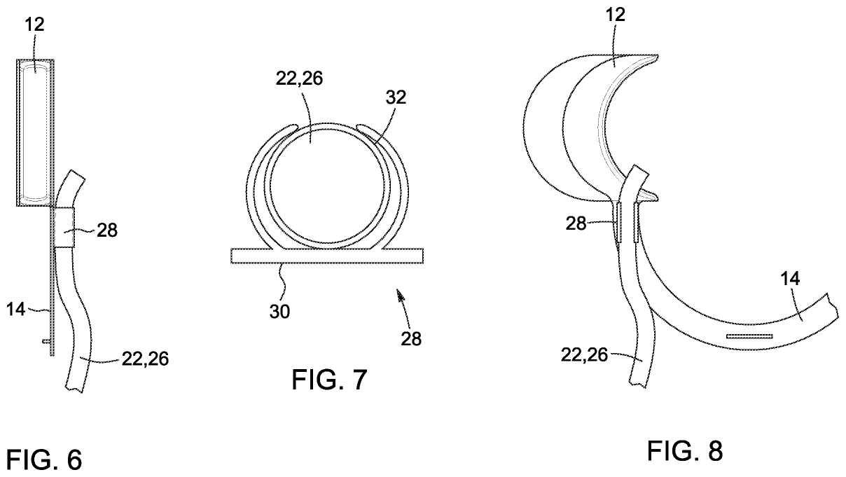

Similarly, and with reference to FIGS. 4 to 8, in some embodiments, the secondary tubes 26 can be connected to the cheek retractor 10 via tube connectors 28. The tube connectors 28 (or tube holders) can be shaped and sized to hold the secondary tubes 26 in place proximate the cheek retractor 10 (e.g., proximate the wings 12). In a possible embodiment, the tube connectors 28 can be a hollow connector superposed and connected to the structure of the cheek retractor 10. For example, and as seen in FIGS. 4 to 6, the tube connectors 28 can be connected to the linking member 14 proximate the wings 12 of the cheek retractor 10, or to the outer portion 17 of the wings 12, although it is appreciated that other configurations are possible.

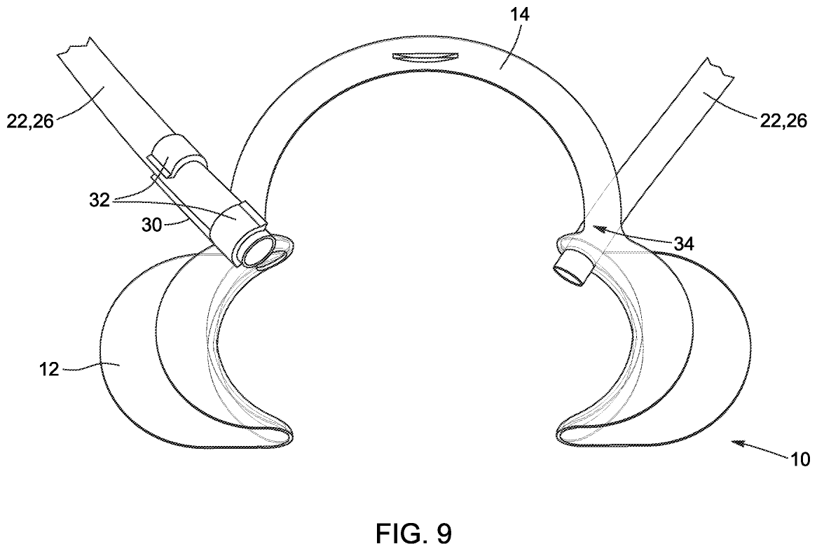

In the embodiment illustrated in FIG. 7, the tube connectors 28 include a substantially flat surface 30 connectable to the cheek retractor 10, and one or more clasping elements 32 adapted to hold one of the tubes 22, e.g., one of the secondary tubes 26. In this embodiment, the tube connectors 28 include two clasping elements 32 spaced along the flat surface 30 (seen in FIG. 9), although any other suitable number and/or configuration of clasping elements 32 can be used. The clasping elements 32 are illustratively connected to the flat surface 30 such that connecting (e.g., superposing) the flat surface 30 to the cheek retractor 10 also positions the secondary tubes 26 proximate the wings 12, and thus proximate the patient's mouth during operation, for example during dental procedures.

In other embodiments, the tube connectors 28 can be inserted in and/or proximate holes defined in a portion of the cheek retractor 10 such that the secondary tubes 26 extend through the structure of the cheek retractor 10 (e.g., through the wings 12). As seen in FIGS. 9 and 10, at least one of the wings 12 of the cheek retractor 10 can be provided with a hole 34 in a bottom portion thereof and through which the secondary tubes 26 can be inserted. More specifically, in this embodiment, the hole 34 can be defined through the central portion 17 of the wing 12, although holes defined through the inner and/or outer portions can also be used to hold the tubes. The secondary tubes 26 can be shaped and sized to cooperate with the holes such that inserting the tubes therethrough connects the tubes to the cheek retractor 10 without requiring additional support and/or connection components. However, it is appreciated that connection components (e.g., tube connectors 28) can engage the secondary tubes 26 on either side of the hole to further secure the tubes 26 with the cheek retractor 10.

The secondary tubes 26 can be connected to the cheek retractor 10 at a predetermined angle to increase the suction provided by the suction device, via the connection assembly. In an exemplary embodiment, the secondary tubes 26 are connected to the cheek retractor 10 on opposite sides thereof (e.g., proximate respective wings 12) in a manner defining a substantially 90-degree angle with an inner surface of the corresponding wing 12 (e.g., the surface of the central portion 17). However, it should be noted that the secondary tubes 26 can be positioned in a manner defining any other suitable angle, or be pivotally connected to the cheek retractor 10 to allow adjustment of the tubes during operations, for example.

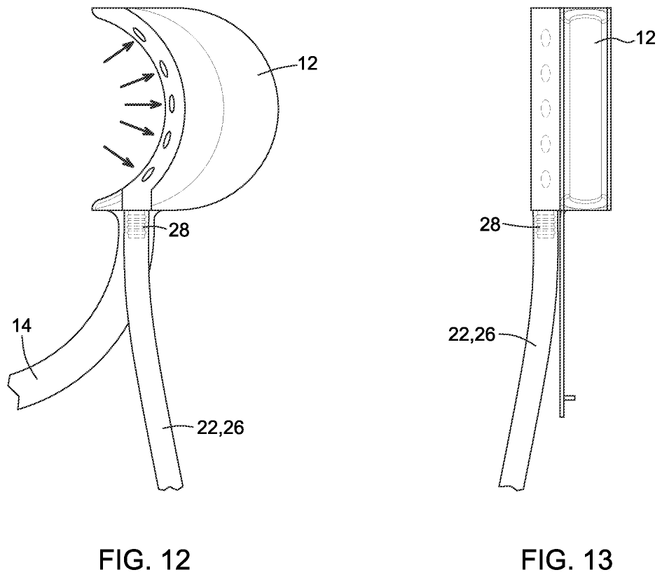

In some embodiments, and as seen in FIGS. 12 and 13, the tube connectors 28 can include an elongate body 28a adapted to extend along an edge of the wing 12, such as along the outer portion 15, for example. In this embodiment, the tube connector 28 can include additional apertures 35 dispersed along a length of the elongate body 28a. Therefore, suction can be achieved at different locations within and around the patient's mouth, thus increasing efficiency of the suctioning feature of the present device. Alternatively, or additionally, the apertures 35 can be defined through the wings 12 themselves or added along a separate strip of material which is connected to the wings 12 via any suitable manner (e.g., adhesive, fasteners, etc.).

In should be noted that the tubes and connectors of the connection assembly 20 can have any suitable shape, size, length and/or configuration, and be made of any suitable material. For example, the tubes can be made of silicone, or any other flexible and light weight material. In an exemplary embodiment, the main tube 24 can have an inner diameter of 10 mm and a length of about 60 cm. The outer diameter of the lower branch of the Y-shaped connector 27 can correspondingly have an outer diameter of about 10 mm to provide a sealed connection with the main tube 24. Similarly, the secondary tubes 26 can have an inner diameter corresponding to an outer diameter of the two upper branches of the Y-shaped connector 27 to define a sealed connection when connected together. In this embodiment, the inner diameter of the secondary tubes 26 and outer diameter of the upper branches of the Y-shaped connector 27 are about 7 mm, although other configurations are possible. The secondary tubes 26 can also have any suitable length, such as 10 cm, 20 cm, 30 cm or 60 cm, for example. The secondary tubes 26 can each have the same length or have different lengths.

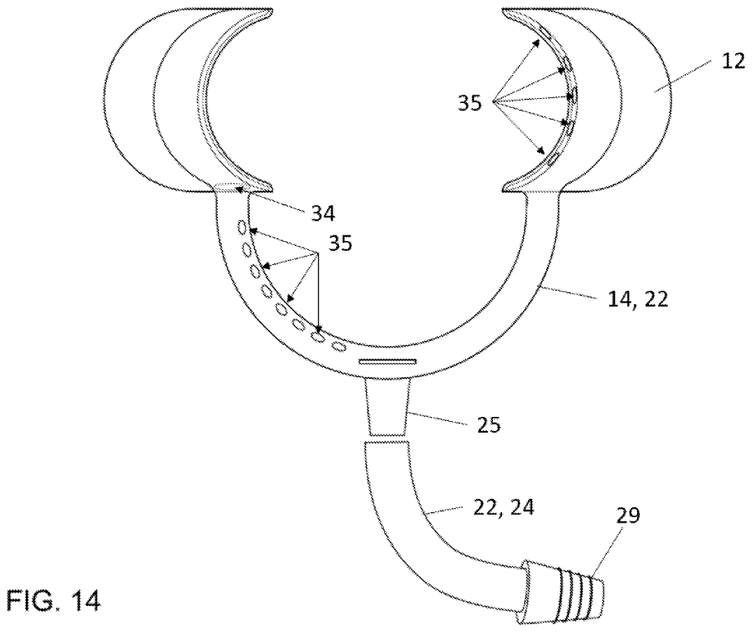

Referring to FIG. 14, a possible alternative embodiment of the cheek retractor device 10 is illustrated. In this embodiment, the linking member 14 extending between the pair of wings 12 is generally hollow such that the linking member 14 can be a tube component 22. The linking member 14 can be connected directly to the main tube 24 via the junction point 25 such that fluids (e.g., airborne particles) can be suctioned from the patient's mouth (e.g., in and around the mouth), into the hollow linking member 14 and finally into the main tube 24. In some embodiments, the linking member 14 can be provided with additional apertures 35 for creating suction at various different locations along the length of the linking member 14. Alternatively, or additionally, the linking member 14 can include holes 34 proximate at least one wing 12 to create suction proximate the cheeks of the patient (i.e., proximate the cheeks being retracted by the device 10).

As may now better be appreciated, the above-described cheek retracting device, and corresponding components, provide substantial improvements over known prior art in that, by virtue of its design and components, as explained herein, and the particular configuration of the cheek retracting device and/or component(s)/accessory(ies) thereof according to the present system, it enables to carry out dental procedures, among others, for a patient in a more efficient, more precise, more accurate, more reliable, more adjustable, more versatile, more adaptable, more impactful, more strategic, and/or more desirable manner (e.g., depending on the circumstances, and the intended results, etc.), compared to what is possible with respect to other known conventional cutting assemblies/tools and/or methods.

Indeed, as previously explained, and depending on the different possible embodiments, the present device advantageously enables to: a) provide a cleaner working environment for the patient, the professional (e.g., the dentist) and each subsequent patient by reducing the amount of airborne particles; b) improve the user's visibility during procedures; c) enable the user to use both hands without having to hold additional equipment to maintain the patient's mouth open; d) provide integrated suction proximate the patient's mouth at one or more locations; e) create an efficient and clear work area in and around the patient's mouth; h) etc.

Of course, and as can be readily understood by a person skilled in the art, the suction device should not be limited by the possible embodiments set forth in the examples, but should be given the broadest interpretation consistent with the description as a whole.

The present disclosure intends to cover and embrace all suitable changes in technology. The scope of the present disclosure is, therefore, described by the appended claims rather than by the foregoing description. The scope of the claims should not be limited by the implementations set forth in the examples, but should be given the broadest interpretation consistent with the description as a whole.

As used herein, the terms “coupled”, “coupling”, “attached”, “connected” or variants thereof as used herein can have several different meanings depending in the context in which these terms are used. For example, the terms coupled, coupling, connected or attached can have a mechanical connotation. For example, as used herein, the terms coupled, coupling or attached can indicate that two elements or devices are directly connected to one another or connected to one another through one or more intermediate elements or devices via a mechanical element depending on the particular context.

In the present disclosure, an embodiment is an example or implementation of the perforation blade. The various appearances of “one embodiment,” “an embodiment” or “some embodiments” do not necessarily all refer to the same embodiments. Although various features may be described in the context of a single embodiment, the features may also be provided separately or in any suitable combination. Conversely, although the robot cell may be described herein in the context of separate embodiments for clarity, it may also be implemented in a single embodiment. Reference in the specification to “some embodiments”, “an embodiment”, “one embodiment”, or “other embodiments”, means that a particular feature, structure, or characteristic described in connection with the embodiments is included in at least some embodiments, but not necessarily in all embodiments.

In the above description, the same numerical references refer to similar elements. In addition, for the sake of simplicity and clarity, namely so as to not unduly burden the figures with several references numbers, not all figures contain references to all the components and features, and references to some components and features may be found in only one figure, and components and features of the present disclosure which are illustrated in other figures can be easily inferred therefrom. The embodiments, geometrical configurations, materials mentioned and/or dimensions shown in the figures are optional, and are given for exemplification purposes only.

Furthermore, although the various exemplary embodiments described herein may be used in relation with dental equipment (e.g., tools used by dentists), for example, it is understood that it may be used with other types of tools and/or for other purposes. For this reason, the terms “cheek retractor”, “cheek retracting device” or any other similar expressions, as used herein should not be taken as to limit the scope of the present disclosure as being used with dental equipment in particular. It should be understood that these terms should, in the context of the present disclosure, encompass all other types of equipment with which the described embodiments could be used and may be useful.

In addition, although the optional configurations as illustrated in the accompanying drawings comprise various components and although the optional configurations of the cheek retractor as shown may consist of certain configurations as explained and illustrated herein, not all of these components and configurations are essential and thus should not be taken in their restrictive sense, i.e. should not be taken as to limit the scope of the present disclosure. It is to be understood that other suitable components and cooperations thereinbetween, as well as other suitable configurations may be used for the cheek retractor, and corresponding parts, as briefly explained, and as can be easily inferred herefrom, without departing from the scope of the disclosure.

1. A cheek retracting device for maintaining cheeks and lips retracted, comprising:
a cheek retractor comprising:
a pair of wings adapted to engage a mouth of a person on either side thereof, each wing comprising an inner portion adapted to extend along an inner side of the cheek, and an outer portion adapted to extend over a periphery of the mouth and engage an outer side of the cheek; and
a resilient linking element connected to and extending between the pair of wings, the resilient linking element being adapted to enable movement of the pair of wings relative to one another and bias the wings in a spaced-apart position to maintain the cheeks and lips retracted when the wings are engaged therewith;
a connection assembly adapted to connect the cheek retractor to a suction device, the connection assembly comprising:
one or more hollow tubes operatively connected to and extending between the suction device and the cheek retractor to enable suction of fluids and airborne particles proximate at least one of the pair of wings and the resilient linking element.

2. The cheek retracting device of claim 1, wherein the resilient linking element is connected to and extends between the outer portions of the pair of wings.

3. The cheek retracting device of claim 1, wherein the one or more hollow tubes comprises a main tube operatively connected to the suction device and one or more secondary tubes having a first end fluidly connected to the main tube, the one or more secondary tubes having a second end provided with a suction aperture positioned proximate one of the wings, where, during operation of the suction device, the suction aperture provides suction to a surrounding area.

4. The cheek retracting device of claim 1, wherein the connection assembly comprises a generally conical and rigid connector adapted to fluidly connect the main tube to the suction device.

5. The cheek retracting device of claim 4, wherein an outer diameter of the rigid connector is substantially the same as the inner diameter of the main tube.

6. The cheek retracting device of claim 3, wherein the connection assembly comprises a junction point having a plurality of connection ports, the main tube and secondary tubes being connected to respective connection ports to establish fluid communication between the main tube and the secondary tubes.

7. The cheek retracting device of claim 3, wherein the connection assembly comprises one or more tube connectors adapted to connect the one or more secondary tubes to the cheek retractor.

8. The cheek retracting device of claim 7, wherein each tube connector comprises a back surface connectable to the cheek retractor, and further comprises one or more clasping elements extending from the back surface and configured to hold the second end of the secondary tubes.

9. The cheek retracting device of claim 8, wherein the back surface is connected to the resilient linking element proximate one of the wings such that the clasping element is adapted to hold the second end of the secondary tube proximate the mouth of the person.

10. The cheek retracting device of claim 8, wherein the clasping elements are generally circular, and wherein an inner diameter of the clasping elements is substantially the same as an outer diameter of the secondary tube.

11. The cheek retracting device of claim 7, wherein the tube connector comprises an elongate body connected to and extending along the outer portion of the wing, the elongate body being hollow and comprising a plurality of apertures provided therealong, the secondary tube being fluidly connected to the tube connector to provide suction to the surrounding environment via each one of the plurality of apertures.

12. The cheek retracting device of claim 7, wherein the tube connectors comprise a substantially conical connection port, and wherein the secondary tube is fluidly connectable to the conical connection port.

13. The cheek retracting device of claim 3, wherein at least one of the wings of the cheek retractor includes a hole defined through the outer portion and being adapted to receive one of the secondary tubes to position the second end thereof proximate the mouth of the person.

14. The cheek retracting device of claim 3, wherein the secondary tubes are positioned proximate the wings of the cheek retractor in a manner such that the second end of the secondary tube and the edge of the wing define an angle between about 75 and 100 degrees.

15. The cheek retracting device of claim 1, wherein the hollow tubes of the connection assembly are made of silicone.

16. The cheek retracting device of claim 1, wherein the hollow tubes are releasably connectable to the cheek retractor.

17. A dental apparatus for insertion within a person's mouth, in combination with a suction device, the dental apparatus comprising:
a cheek retractor having a pair of arcuate wings shaped and adapted to conform to at least a portion of a periphery of a person's mouth; and a resilient linking element connected to and extending between the pair of arcuate wings, the resilient linking element being adapted to enable movement of the pair of wings relative to one another, where the arcuate wings are displaceable toward one another for insertion within the mouth, the resilient linking element being adapted to bias the arcuate wings to a spaced-apart position, where the arcuate wings are adapted to apply an outwardly oriented force on the cheeks to maintain the cheeks and lips retracted; and
a connection assembly having one or more tubing elements connected to the cheek retractor proximate the arcuate wings, the tubing elements being operatively connected to the suction device, with the suction device being operable to provide suction to a surrounding area of the cheek retractor via the tubing elements.

18. The cheek retracting device of claim 17, wherein the tubing elements have a free end positioned proximate the arcuate wings, and wherein the free end comprises a suction port configured to provide suction to the surrounding area of the suction port during operation of the suction device.

19. The cheek retracting device of claim 17, wherein the free end is adapted to extend through a thickness of the arcuate wing.

20. The cheek retracting device of claim 17, wherein the tubing elements are releasably connected to the cheek retractor.
